(19) United States
(12) Patent Application Publication
(10) Pub. No.: US 2023/0212654 A1
(43) Pub. Date: Jul. 6, 2023

(54) LIQUID CHROMATOGRAPHY METHOD FOR SIMULTANEOUSLY DETECTING MULTIPLE MICRORNAS BASED ON DUPLEX-SPECIFIC NUCLEASE (DSN) CYCLIC AMPLIFICATION TECHNOLOGY

(71) Applicant: JIANGSU UNIVERSITY OF SCIENCE AND TECHNOLOGY, Zhenjiang (CN)

(72) Inventors: Wei SHEN, Zhenjiang (CN); Jun SUN, Zhenjiang (CN); Tong QI, Zhenjiang (CN); Sheng TANG, Zhenjiang (CN); Chang SONG, Zhenjiang (CN)

(21) Appl. No.: 17/797,393

(22) PCT Filed: Feb. 7, 2021
(86) PCT No.: PCT/CN2021/075728

§ 371 Date: Aug. 3, 2022

(30) Foreign Application Priority Data
Feb. 7, 2020 (CN) 202010082179.5

(51) Int. Cl. C12Q 1/6834

(52) U.S. Cl. CPC C12Q 1/6834; C12Q 2525/207; C12Q 2563/107; C12Q 2565/137

(57) ABSTRACT

A liquid chromatography method for simultaneously detecting multiple microRNAs based on a duplex-specific nuclease (DSN) cyclic amplification technology comprises the following steps: designing a fluorophore-modified single-stranded DNA probe according to a target microRNA to be detected and loading the probe onto a surface of a streptavidin-coated magnetic bead (MB) to serve as a detection probe; adding a target microRNA sample to be detected and DSN to the detection probe, fully mixing the same, and incubating the mixture; after the incubation, completely removing the magnetic bead and the unreacted DNA probe to obtain a separated solution; and injecting the separated solution into a high-performance liquid chromatography system for separation and quantification.

### FIELD OF TECHNOLOGY

The present disclosure relates to a liquid chromatography method for simultaneously detecting multiple microRNAs based on a duplex-specific nuclease (DSN) cyclic amplification technology and belongs to the technical field of nucleic acid detection.

### BACKGROUND

At present, it is widely believed that microRNAs (miRNAs) are a series of endogenous non-coding small ribonucleic acid molecules (18-25 nucleotides (nt)) and play an important role in biological evolution processes of cell differentiation, proliferation, apoptosis, death and the like. Abnormal expression of miRNAs is closely related to occurrence and development of various diseases, especially various types of human cancers, nervous system diseases, viral infections and diabetes. Thus, the miRNAs are considered as valuable biomarkers for these diseases. However, typically one disease may be associated with multiple miRNAs, or abnormal expression of one specific miRNA may be associated with multiple diseases. Therefore, only analyzing a single miRNA biomarker is insufficient to provide compelling evidence for early diagnosis of cancer or monitoring of the therapeutic effect of cancers. To address this challenge, researchers are increasingly focusing on sensitively analyzing multiple miRNAs through developing various detection methods.

So far, methods have generally been designed using two strategies to analyze multiple miRNAs. The first strategy is to design probes with different signals. For example, Ye and coworkers proposed simultaneous quantitative detection of miRNA-21 and miRNA-203 using a fluorescence-Raman dual-signal switchable nanoprobe switch. The strategy is applicable to simultaneous detection of multiple miRNAs. However, the generally provided method for detecting multiple miRNAs cannot be implemented in one test, for example, the same sample needs to be detected several times by using the same instrument, or different miRNAs are detected by using two different instruments. The second strategy separates miRNAs by high-performance liquid chromatography (HPLC). The high-performance liquid chromatography is a highly efficient separation technique. However, since different miRNAs have too small differences, the separation effect is still not ideal. Although Nakayama et al. successfully separated four types of miRNAs, the miRNAs were detected by nanofluidic LC using a tandem mass spectrometry (MS/MS). To distinguish overlapping signals of five miRNAs (retention times are very close), Xu et al. introduced five DNA-peptide probes as markers for an LC-MS/MS analysis of these five miRNAs. However, obtaining data by tandem mass spectrometry is often cumbersome. In addition, the limit of detections of these detection techniques are far inferior to those using genetic techniques. Therefore, performing a highly sensitive analysis of multiple miRNAs in one test is very urgent.

To improve sensitivity of miRNA detection, a number of strategies for amplifying signals have been developed. The strategies comprise thermal cyclic amplification techniques including a real-time PCR amplification (qRT-PCR) technique, a rolling circle amplification technique, a catalytic hairpin assembly technique, etc., and further comprise strand displacement amplification (SDA) techniques including enzymatic SDA and enzyme-free SDA, etc. As a simple and effective strategy, duplex-specific nuclease (DSN)-assisted target recycling signal cyclic amplification has been attempted for detecting miRNAs. In literature reports, researchers have developed a simple, sensitive, and highly selective detection method for analyzing miRNAs by combining excellent separation of magnetic beads (MBs) and DSN-assisted target recycling. Researchers have also attempted to combine the DSN with the LC-MS/MS for miRNA detection. However, it is only applicable to one type of miRNAs because the HPLC cannot efficiently separate the probes involving DSN-assisted amplification

### SUMMARY

In order to solve the technical problems, the present disclosure provides a liquid chromatography method for simultaneously detecting multiple miRNAs based on a duplex-specific nuclease (DSN) cyclic amplification technology. The technique combines a high-performance liquid chromatography method and a DSN cyclic amplification, which can simultaneously detect multiple miRNAs with high sensitivity.

Technical solutions: to achieve the above objective, the present disclosure uses the following technical solutions:

A liquid chromatography method for simultaneously detecting multiple miRNAs based on a DSN cyclic amplification technology comprises the following steps:
a. designing a fluorophore-modified single-stranded DNA probe according to a target miRNA to be detected and loading the probe onto a surface of a streptavidin-coated magnetic bead (MB) to serve as a detection probe;
b. adding a target miRNA sample to be detected and DSN to the detection probe, fully mixing the same, and incubating the mixture;
c. after the incubation, completely removing the magnetic bead and the unreacted DNA probe to obtain a separated solution; and
d. injecting the separated solution into a high-performance liquid chromatography system for separation and quantification.

Preferably,
in step (1), a ratio of a molar amount of a streptavidin binding site coated on the magnetic bead and a molar amount of the DNA probe is (3-5):1.
in step (1), the loading process is performed in a 2xB&W buffer solution and the buffer solution is prepared from Tris-HCl, EDTA, and NaCl.
in step (2), the target miRNA to be detected is selected from a combination of two, three or more of different target miRNAs; and the target miRNA is a miRNA with 18-25 nucleotides.

As a specific embodiment of the present disclosure, the target miRNA is selected from the group consisting of miRNA-122, miRNA-155 and miRNA-21, and the corresponding single-stranded DNA probe in step (1) is selected from the group consisting of P122, P155, and P21:
P122 has a sequence of 5’-biotin-T₉CAAACACCATTGTCACACTCCAC₆-fluorophore-3’(SEQ ID NO: 01);;
P155 has a sequence of 5’-biotin-T₉ACCCCTATCACGATTAGCATTAAT₃-fluorophore-3’(SEQ ID NO: 02);; and P21 has a sequence of 5’-biotin-T^{g}TCAACATCAGTCTGATAAGCTAT₂₅-fluorophore-3’(SEQ ID NO: 03);;
in step (2), the incubation is performed at 36-38° C. for 140-160 min;
in step (3), the magnetic bead and the unreacted DNA probe are completely removed using a permanent magnet; and
in step (4), the high-performance liquid chromatography system uses a C18 reverse phase chromatographic column and a gradient elution mode.

Further preferably, the gradient elution mode is that a proportion of methanol is changed from 10% to 60% in 20 min; and a mobile phase consists of an organic phase and an aqueous phase containing TEAA.

Preferably, the processes of the method are all performed in a dark place.

The present disclosure develops a highly sensitive determination method capable of simultaneously detecting multiple target miRNAs through HPLC-fluorescence by integrating long and short probe-based DSN-assisted target cyclic amplification. In the method, signals of the target miRNAs are enhanced by DSN-mediated amplification in combination with magnetic separation. After the amplification, the trace amount of the target miRNAs is converted into a large number of cleaved DNA probes which are separated by HPLC (a principle is shown in FIG. 1). Innovation of the method is as follows: (1) a problem of efficiently separating miRNAs by HPLC is solved; (2) the method for sensitively detecting miRNAs on a conventional HPLC fluorescent platform is provided for the first time (no expensive MS/MS system is needed); and (3) multiple miRNAs detection is realized in a single run/test. Finally, the practicality and effectiveness of the method are proved by simultaneously detecting miRNA-122, miRNA-155 and miRNA-21 in a serum sample of a patient with cervical cancer.

The present disclosure firstly loads a plurality of biotin and fluorophore-modified single-stranded DNAs with different lengths and base sequences onto the surfaces of the magnetic beads (MBs) as detection probes. As shown in FIG. 1, the DNA probes on the magnetic beads (e.g., P122, P155, and P21) hybridize to match the target miRNAs to form DNA/RNA heteroduplexes. Since DSN has a strong tendency to cleave DNA in DNA-RNA hybrids, thus the DNA probes of the heteroduplexes can be selectively cleaved by the DSN while target miRNA strands remain intact. A direct result is that the cleavage of the DNA probes releases the target miRNA strands back into a solution for the next cycle. Specifically, under the action of the DSN, DNA in the double strand is cleaved, resulting in dissociation of the fluorophore-containing DNA probes with different lengths and release of the target miRNAs, and the released target miRNAs are for the next cycle. The unreacted DNA probes are completely removed using a permanent magnet to minimize a background signal. Thus, after multiple hybridizations and DSN cleavage cycles over a sufficiently long incubation time, a large number of DNA cleaved probes and fluorescent labels are obtained. After the incubation, only three different lengths of DNA cleaved probes containing fluorescent labels are remained in the solution by magnetic separation, while the MBs and the unreacted DNA probes are completely removed together using a permanent magnet. At this time, fluorescence signals come from three types of the DNA probes. The mixed solution is introduced into an HPLC system to separate the cleaved DNA probes from the fluorescent labels. In addition, a TEM image of streptavidin-coated MBs is shown in FIG. 1. It can be seen that the mean diameter of the MBs is about 300 nm and the MBs are morphologically regular and uniform in size. A light-colored profile coating of a streptavidin layer about 20-nm thick can be clearly observed on the surfaces of the MBs. FIG. 2 shows that the retention time differs when different base tails (cytosine, thymine, and adenine) of different lengths are attached to the DNA probe. Therefore, the retention time can be controlled by changing the length and base type of nucleotides. FIG. 2A shows that the retention time differs when different base tails (cytosine 25 (C25), thymine 25 (T25) and adenine 25 (A25)) are attached to the DNA probe. As can be seen from FIG. 2A, the retention time is C25<A25< T25. Due to the limitations of synthetic techniques, a guanine (G) orthologous nucleotide larger than 6-mers cannot be synthesized. Thus, compared to the retention time of C6 and a shorter T3 (FIG. 2B), the retention time of G6 is very close to that of C6. Generally, T has a longer retention time due to a stronger electrostatic interaction between T and an ion pair reagent and a stronger hydrophobic interaction between the ion pair and an RP column. The interactions will increase with the length of the base sequence as shown in FIG. 2C. Therefore, the retention time can be controlled by changing the length of the nucleotides. C and G have shorter retention time as shown in FIG. 2B. In view of cost and separation efficiency, T25 is selected as a tail of a long DNA probe, and C6 and T3 are selected as a tail of a short DNA probe. The retention time in a chromatogram varies since the cleaved probes have different lengths and base sequences: the shorter DNA probe P122 (with sequence C6) has a retention time of 5.4 min, the P155 (with sequence T3) has a retention time of 6.4 min, and the longer DNA probe P21 (with sequence T25) has a retention time of 6.9 min, thereby simultaneously detecting multiple miRNAs. The method has a limit of detection (LOD) of miRNA-122 at 0.39 fM, miRNA-155 at 0.30 fM, and miRNA-21 at 0.26 fM, and has a linear range of the three miRNAs all at 1.0 fM to 10 pM (as shown in FIG. 5). A correlation between a peak area and a miRNA concentration is shown in FIG. 5. Calibration curves show that the peak area and logarithms of the concentrations of three target miRNAs are separately linearly correlated within the range of 1.0 fM to 100 pM, and the linear coefficients (r²) are separately 0.991, 0.994, and 0.996. The LOD of the method for the three miRNAs is calculated by three times of standard deviation of background signals. The LODs of miRNA-122, miRNA-155 and miRNA-21 are 0.39 fM, 0.30 fM, and 0.26 fM respectively (calculated according to LOD=3NQ/I, where Q is the sample size, N is the noise level, and I represents the fluorescence signal). The relative standard deviation (RSD) is less than 5% over the whole linear range. The established method is successfully used to detect miRNA-122, miRNA-155, and miRNA-21 in serum samples of patients with cervical cancer, lupus erythematosus, and ovarian cancer, and healthy people (see Tables 1-4 and FIG. 7). It is inferred that the method is suitable for two, three or more miRNAs (e.g. a combination of two or more of miRNA-141, let-7a, let-7b, let-7c, let-7d, let-7e, let-7f, let-7i, miRNA-200, miNA-203, miRNA-223, miRNA-16, miRNA-125b, miRNA-199a, miRNA-182-5p, miRNA-210, miRNA-200b, miRNA-200c, miRNA-429, hsa-miRNA-20a, and hsa-miRNA-20b, or a combination with the aforementioned miRNAs), that is, a combination of 2-24 and more combinations.

In order to verify the feasibility of a determination principle, miRNAs, DNA probes and DSN in different proportions are mixed and incubated according to an optimal detection condition. As shown in FIG. 3, in the absence of the target miRNAs, little fluorescence signal is detected regardless of the presence of DSN (FIGS. 3a and b). In the presence of only the target miRNAs and the DNA probes, a negligible background signal is observed (FIG. 3c), probably due to the slight separation of the DNA/RNA heteroduplexes from the MBs. The fluorescence signals are observed only when the target miRNAs, DSN and the DNA probes coexist in the solution (FIGS. 3d, e, f, and g). The above results clearly show that the provided measurement method is feasible for simultaneous highly-sensitive detection of miRNA-122, miRNA-155, and miRNA-21.

The experiment of the present disclosure proves the feasibility of simultaneously detecting multiple miRNAs in practical samples by using an HPLC fluorescent platform. The separation of signals of different miRNAs is realized by introducing long and short DNA probes and using the HPLC. The introduction of the DSN promotes the successful use of the isothermal target cyclic amplification method, successfully solves the problem of the low sensitivity of a conventional HPLC fluorescence detection, and simultaneously ensures high selectivity of the method to the target miRNAs (FIG. 6). Fluorescence responses of other mismatched miRNAs are compared with that of the target miRNAs under the same optimal experimental detection condition. FIG. 6 shows the responses of the target miRNAs, single base mismatched miRNAs, double base mismatched miRNAs, and completely mismatched miRNAs. No obvious interference is observed even when a concentration of an interferent is 100-fold higher than those of the target miRNAs. The peak areas of single base, double base, and completely mismatched miRNAs are negligible, while the presence of target miRNA-122, miRNA-155, and miRNA-21 significantly increases the fluorescence signals. These results indicate that the method is very effective for detecting the target miRNAs. Meanwhile, FIG. 7 shows a chromatogram of an original sample and a spiked serum sample after the method is used. It illustrates that the determination method conveniently simultaneously detects multiple miRNAs in a single run. In addition, the analysis method of the present disclosure will facilitate highly sensitive and highly selective analysis of biological macromolecules (e.g. nucleic acids) based on the conventional HPLC method, and greatly improve the separation efficiency of the DNA probes after cleavage by introducing more types of bases (A and G) as the tails of the DNA probe. Therefore, more target miRNAs can be detected simultaneously. In addition, the use of new amplification methods (such as LH-PCR) in combination with the HPLC has a potential for analyzing length heterogeneity (LH) of nucleic acids. Compared with the prior art, the beneficial effects of the method are simultaneous detection of multiple miRNAs by using a common liquid chromatograph, low instrument cost, convenient operation, high sensitivity, low limit of detection and wide linear range. As shown in the example: miRNA-122 has the limit of detection of 0.39 fM, miRNA-155 is 0.30 fM, and miRNA-21 of 0.26 fM with a linear range all of 1.0 fM to 10 pM. The established method can be successfully used to detect miRNA-122, miRNA-155, and miRNA-21 in serum samples of patients with lupus erythematosus, cervical cancer, and ovarian cancer, and healthy people.

### BRIEF DESCRIPTION OF THE DRAWINGS

FIG. 1 is a mechanism diagram of a high- performance liquid chromatography analysis method for simultaneously detecting miRNA-122, miRNA-155, and miRNA-21 based on a DSN cyclic amplification technology;

FIG. 2A shows chromatograms of DNA probes, the chromatogram of the DNA probes with different base sequences of the same length (25 nt);

FIG. 2B shows chromatograms of DNA probes, the chromatogram of the DNA probes with the same base sequence (cytosine C and guanine G) of different lengths;

FIG. 2C shows chromatograms of DNA probes, the chromatogram of the DNA probes with the same base sequence (thymine T) of different lengths;

FIG. 3 is a graph of fluorescence signals verifying feasibility of simultaneously detecting miRNA-122, miRNA-155, and miRNA-21,

fluorescence signals of (a) blank, (b) 0.4 U DSN, (c) 100 pM miRNA-122+100 pM miRNA-155+100 pM miRNA-21, (d) 100 pM miRNA-122+0.4 U DSN, (e) 100 pM miRNA-155+0.4 U DSN, (f) 100 pM miRNA-21+0.4 U DSN, and (g) 100 pM miRNA-122+100 pM miRNA-155+100 pM miRNA-21+0.4 U DSN, experimental conditions: 100 nM of DNA probes, 25 mM of Mg²⁺, pH 8.0 and incubation at 40° C. for 180 min, and error bars represent standard deviation of three independent experiments;

FIG. 4A shows optimization of experimental conditions for 100 pM of target miRNA-122, miRNA-155 and miRNA-21, and 100 nM of DNA probes: a concentration of DSN from 0.1 U to 0.5 U;

FIG. 4B shows optimization of experimental conditions for 100 pM of target miRNA-122, miRNA-155 and miRNA-21, and 100 nM of DNA probes: a pH value of a buffer solution from 7 to 9;

FIG. 4C shows optimization of experimental conditions for 100 pM of target miRNA-122, miRNA-155 and miRNA-21, and 100 nM of DNA probes: a concentration of mg²⁺ from 15 mM to 35 mM;

FIG. 4D shows optimization of experimental conditions for 100 pM of target miRNA-122, miRNA-155 and miRNA-21, and 100 nM of DNA probes: chromatograms of probes corresponding to different concentrations of mg²⁺;

FIG. 4E shows optimization of experimental conditions for 100 pM of target miRNA-122, miRNA-155 and miRNA-21, and 100 nM of DNA probes: an incubation temperature from 30° C. to 50° C.;

FIG. 4F shows optimization of experimental conditions for 100 pM of target miRNA-122, miRNA-155 and miRNA-21, and 100 nM of DNA probes: incubation time from 60 min to 210 min;

FIG. 5A show calibration curves for three miRNAs: miRNA-122; experimental conditions: 100 nM of DNA probes, 25 mM of Mg²⁺, 0.4 U DSN, pH 8.0 and incubation at 40° C. for 180 min; and error bars represent standard deviation of three independent experiments;

FIG. 5B show calibration curves for three miRNAs: miRNA-155; experimental conditions: 100 nM of DNA probes, 25 mM of Mg²⁺, 0.4 U DSN, pH 8.0 and incubation at 40° C. for 180 min; and error bars represent standard deviation of three independent experiments;

FIG. 5C show calibration curves for three miRNAs: miRNA-21; experimental conditions: 100 nM of DNA probes, 25 mM of Mg²⁺, 0.4 U DSN, pH 8.0 and incubation at 40° C. for 180 min; and error bars represent standard deviation of three independent experiments;

FIG. 6 is a graph for selectively detection of miRNA-122, miRNA-155, and miRNA-21: peak areas for M1 (single base mismatch), M2 (double base mismatch) and NM (complete mismatch), interfering miRNA at a concentration of 10 nM, and each miRNA at a concentration of 100 pM; experimental conditions: 100 nM of DNA probes, 25 mM of Mg²⁺, 0.4 U DSN, pH 8.0 and incubation at 40° C. for 180 min; and error bars represent standard deviation of three independent experiments; and

FIG. 7 shows chromatograms of an original sample and a serum sample containing a certain amount of miRNAs and the established method is used to detect the chromatograms of the original sample and the spiked serum sample; and experimental conditions: 100 nM of DNA probes, 25 mM of Mg²⁺, 0.4 U DSN, pH 8.0 and incubation at 40° C. for 180 min.

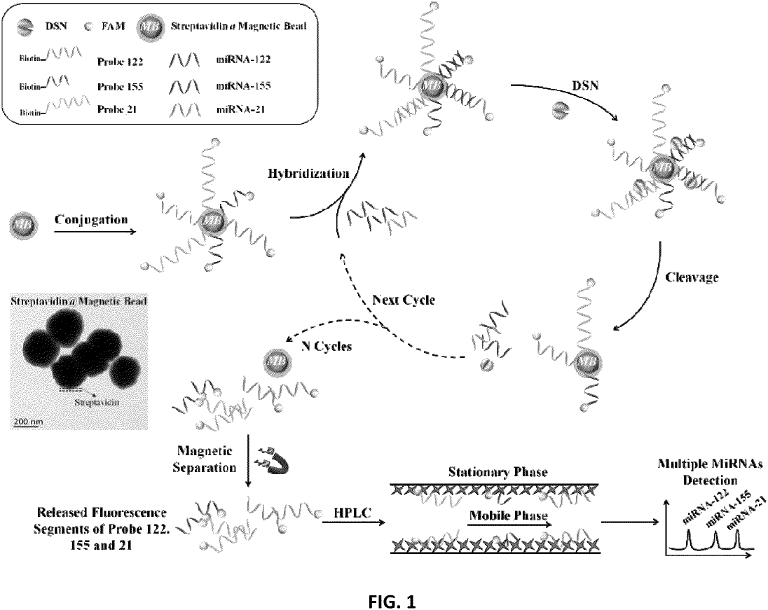

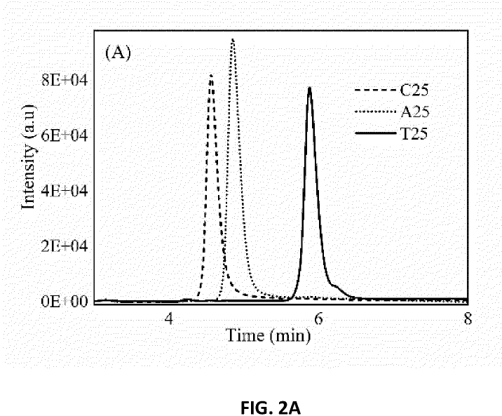

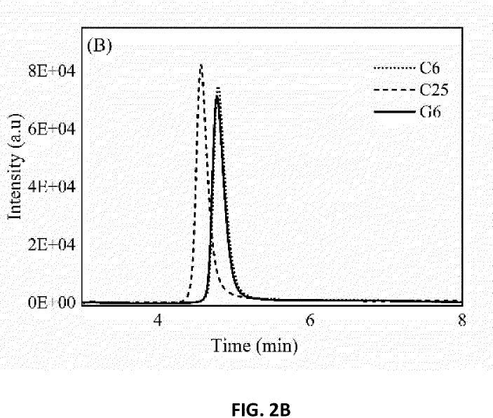

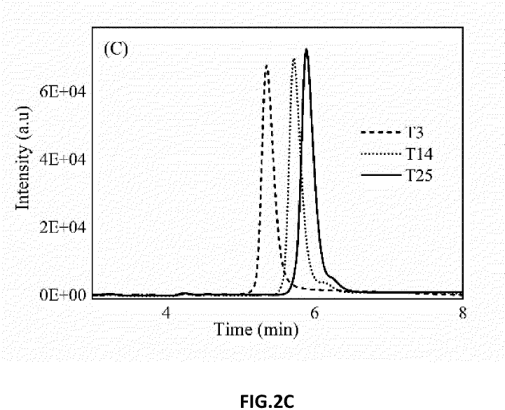

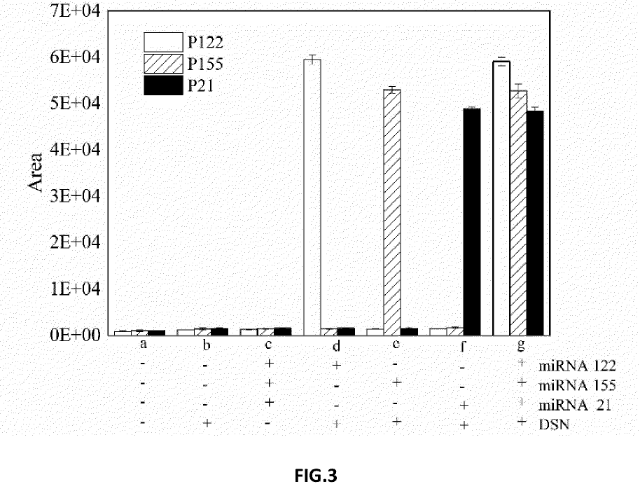

### DETAILED DESCRIPTION OF THE EMBODIMENTS

The method of the present disclosure will be described in more detail below with reference to the accompanying drawings and specific examples.

A liquid chromatography method for detecting multiple microRNAs based on a DSN cyclic amplification technology comprises the following steps:

Firstly adding 40 µL of streptavidin-coated magnetic beads (MBs) with a particle size of 300 nm into a 1.5-mL brown polypropylene centrifuge tube, performing magnetic separation to remove a solvent, and retaining the MBs in the tube; after washing 3 times with a 1xB&W buffer solution, redispersing the MBs in 90 µL of a 2xB&W buffer solution, and adding 5 µL of 10 µM of DNA probes of different lengths (in the example, P122 is 37 nt, P155 is 35 nt, and P21 is 58 nt); gently vortexing at a room temperature for 15 min to ensure adequate binding of biotin on the probes and streptavidin from the MBs performing magnetic separation to obtain a supernatant and fluorescence detection at an excitation wavelength of 495 nm and an emission wavelength of 518 nm; estimating the coupling efficiency between the probes and the MBs according to fluorescence data; wherein it is estimated that there are about 1.16x10⁴ DNA probes (the sum of P122, P155, and P21 coupled to each MB accounts for about 12% of the total capacity of the MBs), and thus, there is a sufficient space for target miRNA hybridization and DSN cleavage; and finally, washing and dispersing the obtained long and short DNA probes and MB conjugates in a hybridization buffer solution, and storing the same at 4° C. for a standby application.

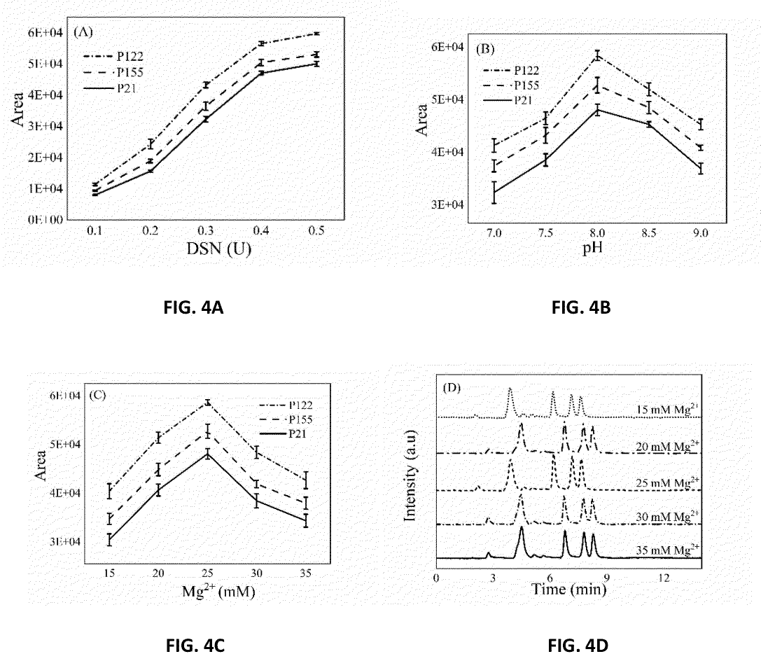

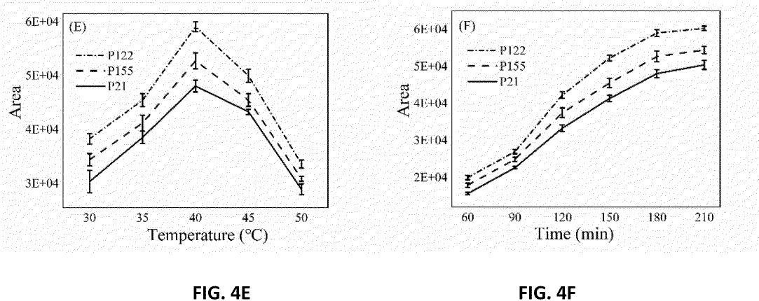

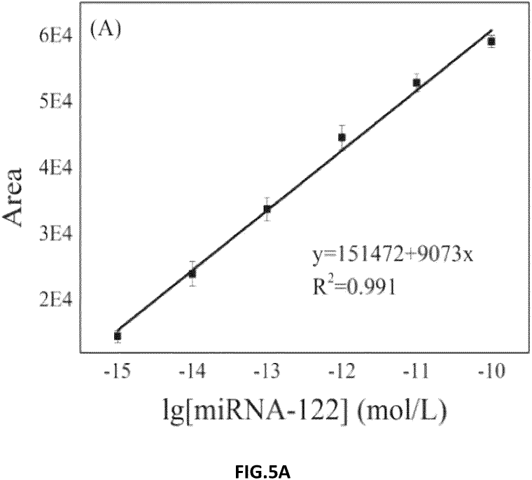

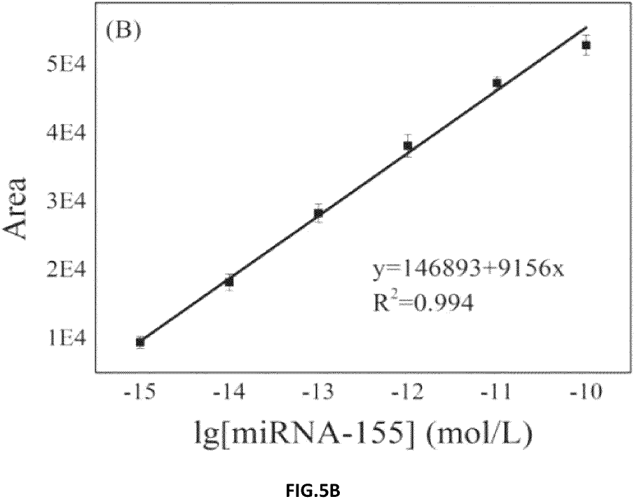

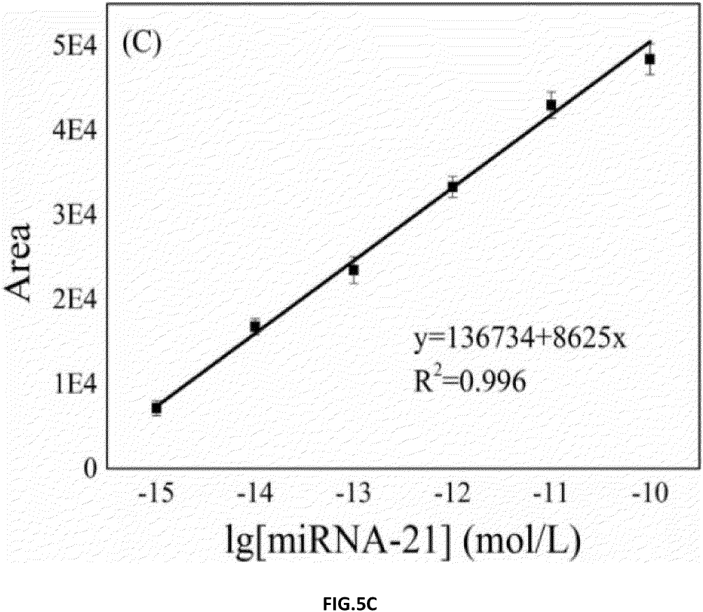

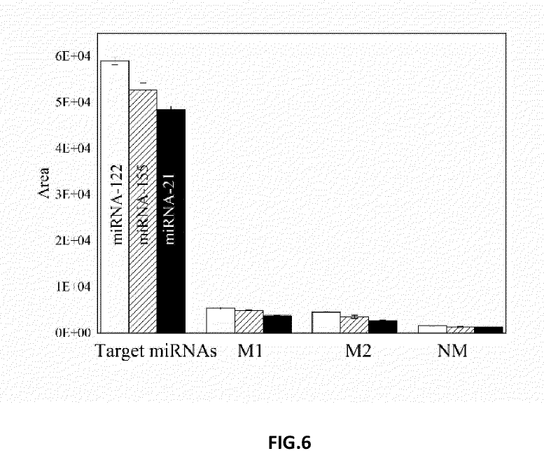

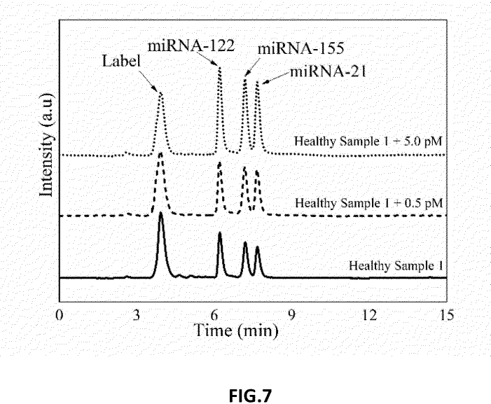

Before the target miRNAs are detected, all experimental conditions are optimized, including conjugation time, the dose of DSN, concentration of Mg²⁺, pH of buffer solution, and incubation temperature and time. The optimization results are shown in FIG. 4.

It can be seen from FIG. 4A that the peak area of the fluorescence signal increases significantly when the dose of DSN is changed from 0.10 U to 0.50 U, while the peak area of the fluorescence signal of P122, P155, and P21 all tends to be stable when DSN > 0.40 U. To improve detection sensitivity and keep costs low maximally, 0.4 U is used as the optimal dose of DSN, which is sufficient to detect miRNAs as low as a femtomolar level. The pH value will strongly affect the cleavage rate of the DNA probes and thus affect measured amplification capacity. To maximally improve the amplification capacity, the pH value of the sample solution is adjusted from 7 to 9. It is reported that the DSN has a working range of 3.5 to 8.5 with the maximum activity at a pH of about 6.6 and the DSN is deactivated when pH value < 3.0 or > 9.0. In addition, the pH value of the solution also has a significant effect on the fluorescence of the fluorescein labels. In an acidic medium, the fluorescein is typically protonated. Instead, the fluorescein is deprotonated in an alkaline medium. Both protonated and deprotonated states change the conformation of the fluorescein, and thus the fluorescence efficiency is reduced. According to the observation of our experiment (FIG. 4B), pH 8.0 is found to be the best choice for the method. The presence of Mg²⁺ is of great significance in promoting the cleavage of the DSN and the efficient hybridization of the miRNA/DNA heteroduplexes. Therefore, the concentration of Mg²⁺ plays a key role in detecting miRNAs. The present disclosure evaluates the effect of the parameter and the results are shown in FIG. 4C and D. It can be seen that the peak area increases with the increase of the content of Mg²⁺, but the peak area decreases significantly when the concentration of Mg²⁺ is above 25 mM. The possible reason is that the DSN activity is reduced at high ionic strength. In view of these results, 25 mM Mg²⁺ is selected as the optimum concentration for the measurement. Another important variable is the incubation temperature of the reaction. The incubation temperature directly affects the hybridization efficiency and the cleavage activity of the DSN. In practical use, a melting temperature should be 10-15° C. higher than a hybridization temperature to achieve high hybridization efficiency while ensuring good selectivity. Under the current experimental conditions, the melting temperatures of miRNA-122, miRNA-155, and miRNA-21 are estimated to be about 53° C., 54° C., and 51° C., separately. Therefore, theoretically, the experiment should be performed below 45° C. The cleavage by the DSN is good at a temperature above 45° C. and has an optimum temperature of 60° C. As shown in FIG. 4E, the peak areas of the three miRNAs reach a maximum at 40° C. Therefore, 40° C. is used as the incubation temperature for the measurement. A series of incubation time from 60 min to 210 min is used in the experiment. FIG. 4F shows that as the incubation time increases from 60 min to 180 min, the peak area of the fluorescence signal increases rapidly, after which the peak areas of the three miRNAs tend to be stable. These results indicate that 180 min of incubation is sufficient to cleave the DNA probes. In view of the reaction efficiency, since no significant difference is observed after a longer incubation time, 180 min is selected as the optimal time for a subsequent experiment.

The method used for optimizing all the examples of the present disclosure is as follows: 44 µL of 100 nM of P122, P155, and P21 are added to a centrifuge tube, followed by 0.4 U of DSN and 5 µL of target miRNAs. Then a short shaking treatment is performed for about 2 s to mix the reaction mixture fully. After incubation at 40° C. for 180 min, MBs are separated with a permanent magnet along with unreacted DNA probes. Finally, the obtained supernatant is injected into a high-performance liquid chromatography system for separation and quantification.

The high-performance liquid chromatography is performed using a Shimadzu LC-20A system equipped with a Shimadzu RP-20A fluorescence detector. Data acquisition and processing are completed using an LCsolution data analysis software (free version). A clarity reverse phase chromatographic column from Phenomenex (50×4.6 mm (internal diameter) and 3 µm particle size) is used for separating miRNAs. A column temperature is maintained at 35° C. A gradient elution mode is used. The parameters of the fluorescence detector are set as an excitation wavelength of 495 nm and an emission wavelength of 518 nm.

The 1xB&W buffer solution required for an experiment at a pH of 7.5 is prepared from 5.0 mM of Tris-HCl, 0.5 mM of EDTA, and 1.0 M of NaCl. The 2xB&W buffer solution required for an experiment at a pH of 7.5 is prepared from 10.0 mM of Tris-HCl, 1.0 mM of EDTA, and 2.0 M of NaCl. The hybridization buffer solution required from an experiment at a pH of 8 is prepared from 50 mM of Tris-HCl and 25 mM MgCl₂.

A stationary phase of the chromatographic column in the experiment is octadecyl (C18). The C18 column is a typical reverse phase (RP) column and commonly used to retain and separate hydrophobic compounds. However, oligonucleotides are strongly polar and difficult to retain in any RP columns. Therefore, 100 mM of TEAA is added as an ion-pairing reagent in a mobile phase to make the retention time of the DNA probes on the column longer.

In order to prevent exposure to light that could adversely affect the fluorescent properties of fluorophores, all the steps related to the fluorophores are performed in aluminium foil-wrapped centrifuge tubes.

The gradient elution mode used in the experiment is that a proportion of methanol is changed from 10% to 60% in 20 min, and a flow rate is 1 mL/min.

The mobile phase in the experiment consists of an organic phase of methanol and an aqueous phase of 100 mM of a TEAA aqueous solution and 5% acetonitrile.

The method of the present disclosure simultaneously detects multiple miRNAs. In the example: miRNA-122 has the limit of detection of 0.39 fM, miRNA-155 of 0.30 fM, and miRNA-21 of 0.26 fM with a linear range all of 1.0 fM to 10 pM. The established method is successfully used to detect miRNA-122, miRNA-155, and miRNA-21 in serum samples of patients with lupus erythematosus, cervical cancer, and ovarian cancer, and healthy people.

The method is used to detect miRNA-122, miRNA-155, and miRNA-21 in serum samples of healthy people, and patients with lupus erythematosus, cervical cancer, and ovarian cancer. Specific examples are as follows:

### Example 1 Detection of miRNA-122, miRNA-155, and miRNA-21 in Serum Samples of Healthy People

The detection results of miRNA-122, miRNA-155, and miRNA-21 in serum samples of healthy people were shown in Table 1. Concentrations of miRNA-122, miRNA-155, and miRNA-21 were detected to be 0.063 pM, 0.057 pM, and 0.046 pM separately in serum sample 1 (healthy volunteers), and 0.067 pM, 0.053 pM, and 0.048 pM separately in serum sample 2 (healthy volunteers). To evaluate the effect of matrices, different concentrations of miRNA-122, miRNA-155, and miRNA-21 were spiked into sample 1 to obtain a good relative recovery of 101.7%-104.7% and a relative standard deviation (RSD) of 2.4 %-4.7%. These results were consistent with those of qRT-PCR. The results clearly demonstrated that the provided method had good practicability for analyzing multiple miRNAs in practical samples.

TABLE 1

Detection of miRNA-122, miRNA-155, and miRNA-21 in serum samples of healthy people

| | miRNA-122 (pM) | | | miRNA-155 (pM) | | | miRNA-21 (pM) | | |
|---|---|---|---|---|---|---|---|---|---|
| Sample | Spiked | The method (RSD%) | Relative recovery (%) | Spiked | The method (RSD%) | Relative recovery (%) | Spiked | The method (RSD%) | Relative recovey (%) |
| Sample 1 | Blank | 0.063 (2.7) | - | Blank | 0.057 (4.5) | - | Blank | 0.046 (3.6) | - |
| Serum of | Blank+ 0.50 | 0.584 (4.7) | 104.1 | Blank+0. 50 | 0.570 (3.1) | 102.4 | Blank+0. 50 | 0.571 (2.4) | 104.7 |
| healthy people | Blank+ 5.00 | 5.151 (2.6) | 101.7 | Blank+5. 00 | 5.076 (2.7) | 100.4 | Blank+5. 00 | 5.131 (2.6) | 101.6 |
| Sample 2 | | | | | | | | | |
| Serum of healthy people | Blank | 0.067 (1.4) | - | Blank | 0.053 (2.7) | - | Blank | 0.048 (2.1) | - |

^{a}Relative recovery=(total concentration-blank concentration)/spiked concentration

### Example 2 Detection of miRNA-155 and miRNA-21 in Serum Samples of Patient with Lupus Erythematosus

The detection results of miRNA-155 and miRNA-21 in serum samples of a patient with lupus erythematosus were shown in Table 2. 0.399 pM of miRNA-155 and 0.034 pM of miRNA-21 were separately detected in the serum samples of the patient with lupus erythematosus. Compared with healthy people, miRNA-155 was significantly overexpressed in the patient with lupus erythematosus. These results were consistent with those of qRT-PCR. The results clearly demonstrated that the provided method had good practicability for analyzing multiple miRNAs in practical samples.

TABLE 2

Detection of miRNA-155 and miRNA-21 in serum samples of patient with lupus erythematosus

| | miRNA-155 (pM) | | | miRNA-21 (pM) | | |
|---|---|---|---|---|---|---|
| Sample | Spiked | The method (RSD%, n=3) | Relative recovery (%) | Spiked | The method (RSD%, n=3) | Relative recovery (%) |
| Serum of patient with lupus erythematosus | Blank | 0.399 (1.2) | - | Blank | 0.034 (1.7) | - |

^{a}Relative recovery=(total concentration-blank concentration)/spiked concentration

### Example 3 Detection of miRNA-155 and miRNA-21 in Serum Samples of Patient with Ovarian Cancer

The detection results of miRNA-155 and miRNA-21 in serum samples of a patient with ovarian cancer were shown in Table 3. 0.090 pM of miRNA-155 and 0.137 pM of miRNA-21 were separately detected in the serum sample of the patient with ovarian cancer. Compared with healthy people, miRNA-21 was significantly overexpressed in the patient with ovarian cancer. These results were consistent with those of qRT-PCR. The results clearly demonstrated that the provided method had good practicability for analyzing multiple miRNAs in practical samples.

TABLE 3

Detection of miRNA-155 and miRNA-21 in serum samples of patient with ovarian cancer

| | miRNA-155 (pM) | | | miRNA-21 (pM) | | |
|---|---|---|---|---|---|---|
| Sample | Spiked | The method (RSD%, n=3) | Relative recovery (%) | Spiked | The method (RSD%, n=3) | Relative recovery (%) |
| Serum of patient with ovarian cancer | Blank | 0.090 (3.7) | - | Blank | 0.137 (3.7) | - |

### Example 4 Detection of miRNA-122, miRNA-155, and miRNA-21 in Serum Samples of Patients with Cervical Cancer

The detection results of miRNA-122, miRNA-155, and miRNA-21 in serum samples of a patient with cervical cancer were shown in Table 4. Concentrations of miRNA-122 were detected to be 0.070 pM and 0.090 pM separately in sample 3 and 4 (patients with cervical cancer), concentrations of miRNA-155 were detected to be 0.209 pM and 0.224 pM separately, and concentrations of miRNA-21 were detected to be 0.115 pM and 0.117 pM separately. These results indicated that miRNA-155 and miRNA-21 were up-regulated in the patients with cancer compared with healthy people. These results were consistent with those of qRT-PCR. The results clearly demonstrated that the provided method had good practicability for analyzing multiple miRNAs in practical samples.

TABLE 4

Detection of miRNA-122, miRNA-155, and miRNA-21 in serum samples of patients with cervical cancer

| | miRNA-122 (pM) | | | miRNA-155 (pM) | | | miRNA-21 (pM) | | |
|---|---|---|---|---|---|---|---|---|---|
| Sample | Spiked | The method (RSD%) | Relative recovery (%) | Spiked | The method (RSD%) | Relative recovery (%) | Spiked | The method (RSD%) | Relative recovery (%) |
| Sample 3 | Blank | 0.070(4.5) | - | Blank | 0.209(3.5) | - | Blank | 0.115(3.9) | - |
| Serum of patient with cervical cancer | Blank+0.50 | 0.578(3.9) | 102.9 | Blank+0.50 | 0.765(4.1) | 102.4 | Blank+0.50 | 0.621(3.3) | 101.2 |
| | Blank+5.00 | 5.283(1.6) | 104.4 | Blank+5.00 | 5.120(3.1) | 98.21 | Blank+5.00 | 5.221(1.1) | 102.2 |
| Sample 4 | | | | | | | | | |
| Serum of patient with cervical cancer | Blank | 0.090(3.7) | - | Blank | 0.224(2.4) | - | Blank | 0.117(2.7) | - |

^{a}Relative recovery=(total concentration-blank concentration)/spiked concentration

### Example 5 Comparison of Different Methods for Detecting miRNAs

The method of the present disclosure was compared with other methods for the detection of multiple miRNAs or methods related to HPLC reported in recent years, and the results were shown in Table 5. The method for analyzing miRNAs using fluorescence detection was highly selective but not sensitive (Wang, R.; Xu, X.; Li, X.; Zhang, N.; Jiang, W. pH-responsive ZnO nanoprobe mediated DNAzyme signal amplification strategy for sensitive detection and live cell imaging of multiple microRNAs. Sens. Actuators, B. 2019, 293, 93-99.Jie, G.; Zhao, Y.; Wang, X.; Ding, C. Multiplexed fluorescence detection of microRNAs based on novel distinguishable quantum dot signal probes by cycle amplification strategy. Sens. Actuators, B. 2017, 252, 1026-1034). The electrochemiluminescence (ECL)-based miRNA measurement methods showed high sensitivity, but had disadvantages such as synthesizing complex materials (Feng, X.; Gan, N.; Zhang, H.; Li, T.; Cao, Y.; Hu, F.; Jiang, Q. Ratiometric biosensor array for multiplexed detection of microRNAs based on electrochemiluminescence coupled with cyclic voltammetry. Biosens. Bioelectron. 2016, 75, 308-314.Peng, L.; Zhang, P.; Chai, Y.; Yuan, R. Bi-directional DNA Walking Machine and Its Application in an Enzyme-Free Electrochemiluminescence Biosensor for Sensitive Detection of MicroRNAs. Anal. Chem. 2017, 89 (9), 5036-5042). The HPLC-MS/MS-based analysis showed high selectivity but often involved tedious data analysis (Kuang, Y; Cao, J.; Xu, F.; Chen, Y. Duplex-Specific Nuclease-Mediated Amplification Strategy for Mass Spectrometry Quantification of miRNA-200c in Breast Cancer Stem Cells. Anal. Chem. 2019, 91 (14), 8820-8826. Liu, L.; Xu, Q.; Hao, S.; Chen, Y. A Quasi-direct LC-MS/MS-based Targeted Proteomics Approach for miRNA Quantification via a Covalently Immobilized DNA-peptide Probe. Sci. Rep. 2017, 7 (1), 5669). Furthermore, these analyses relied on the detection of peptide chains and indirectly relied on the insertion of targeted proteomics into miRNAs for quantification. More importantly, most of these analytical methods cannot detect multiple miRNAs in a single run. Furthermore, it can be seen that the limit of detection (LOD) measured in the experiment is much lower than the LOD reported in the literature. Although Nakayama et al. identified more than a dozen human cellular miRNAs in a single untargeted nanoflow LC-MS/MS, the method still requires more sophisticated instruments and has a lower LOD than other genetics methods (Nakayama, H.; Yamauchi, Y.; Taoka, M.; Isobe, T. Direct Identification of Human Cellular MicroRNAs by Nanoflow Liquid Chromatography-High-Resolution Tandem Mass Spectrometry and Database Searching. Anal. Chem. 2015, 87 (5), 2884-2891). It can be seen that the method of the present disclosure has an advantage of better simultaneously detecting multiple miRNAs.

TABLE 5

Comparison of different methods for detecting miRNAs

| analytical methods | target | sample | mechanism | single run/test | LOD | linear range |
|---|---|---|---|---|---|---|
| fluorescence | miRNA-21 | cells | pH-responsive ZnO nanoprobe mediated DNAzyme | no | 54 pM | 100 pM-30 nM |
| | miRNA-373 | | | | 38 pM | 100 pM-20 nM |
| | miRNA-141 | cells | quantum dot signal probes with cycle amplification strategy | no | 1.5 pM | 5.0 pM-50.0 |
| | miRNA-21 | | | | 1.5 pM | nM |
| ECL | miRNA-21 | serum | Ru(bpy)₃²⁺- Silica@Poly-L-lysine-Au NPs | no | 6.3 fM | 0.02-150 pM |
| | miRNA-141 | | | | 8.6 fM | 0.03-150 pM |
| | miRNA-21 | PBS | bi-directional DNA walking machine | no | 1.51 fM | 5 fM-500 pM |
| | miRNA-155 | | | | 1.67 fM | |
| surface-enhanced Raman scattering | miRNA-21 | cells | electromagnetic hot spots vis target-mediated nanoparticle dimerization strategy | yes | 1 pM | 1.0 pM- 10 nM |
| | miRNA-155 | | | | 1 pM | |
| LC-MS/MS | hsa-let7 family hsa-miRNA-15b, 16, 21, 23a, 24, 25, 27a, 27b, 30b, 30c, 98, 106b, 125b, 365a | cells | nano-flow LC-high-resolution tandem MS and RNAsequence database searching | yes | 300 amol | 300 amol to 300 fmol |
| | miRNA-21 | cells | quasi-targeted proteomics approach | no | 1 pM ( LOQ) | 1 pM-100 nM |
| | miRNA-let7a | | | | | |
| | miRNA-200c | | | | | |
| | miRNA-125a | | | | | |
| | miRNA-15b | | | | | |
| | miRNA-200c | cells | DSN mediated amplification | — | 1 fM (LOQ) | 1 fM-200 fM |
| | miRNA-21 | cells | proteomics approach via a covalently immobilized DNA peptide probe | — | 5 pM (LOQ) | 5 pM-10 nM |
| HPLC-fluorescence | miRNA-122 | serum | long and short probes based DSN mediated amplification | yes | 0.39 fM | 1.0 fM-100 pM |
| | miRNA-155 | | | | 0.30 fM | 1.0 fM-100 pM |
| | miRNA-21 | | | | 0.26 fM | 1.0 fM-100 pM |

What is claimed is:

1. A liquid chromatography method for simultaneously detecting multiple microRNAs based on a duplex-specific nuclease (DSN) cyclic amplification technology, comprising the following steps:
(1) designing a fluorophore-modified single-stranded DNA probe according to a target microRNA to be detected and loading the probe onto a surface of a streptavidin-coated magnetic bead (MB) to serve as a detection probe;
(2) adding a target microRNA sample to be detected and DSN to the detection probe, fully mixing the same, and incubating the mixture;
(3) after the incubation, completely remove the magnetic bead and the unreacted DNA probe to obtain a separated solution; and
(4) injecting the separated solution into a high-performance liquid chromatography system for separation and quantification.

2. The liquid chromatography method for simultaneously detecting multiple miRNAs based on a DSN cyclic amplification technology according to claim 1, wherein in step (1), a ratio of a molar amount of a streptavidin binding site coated on the magnetic bead and a molar amount of the DNA probe is (3-5):1.

3. The liquid chromatography method for simultaneously detecting multiple microRNAs based on a DSN cyclic amplification technology according to claim 1, wherein in step (1), the loading process is performed in a 2×B&W buffer solution and the buffer solution is prepared from Tris-HCl, EDTA, and NaCl.

4. The liquid chromatography method for simultaneously detecting multiple microRNAs based on a DSN cyclic amplification technology according to claim 1, wherein in step (2), the target microRNA to be detected is selected from a combination of two, three or more of different target miRNAs; and the target miRNA is a miRNA with 18-25 nucleotides.

5. The liquid chromatography method for simultaneously detecting multiple microRNAs based on a DSN cyclic amplification technology according to claim 1, wherein in step (2), the target miRNA is selected from the group consisting of miRNA-122, miRNA-155, and miRNA-21, and the corresponding single-stranded DNA probe in step (1) is selected from the group consisting of P122, P155, and P21.

6. The liquid chromatography method for simultaneously detecting multiple miRNAs based on a DSN cyclic amplification technology according to claim 1, wherein in step (2), the incubation is performed at 36-38° C. for 140-160 min.

7. The liquid chromatography method for simultaneously detecting multiple microRNAs based on a DSN cyclic amplification technology according to claim 1, wherein in step (3), the magnetic bead and the unreacted DNA probe are completely removed using a permanent magnet to reduce a background interference.

8. The liquid chromatography method for simultaneously detecting multiple microRNAs based on a DSN cyclic amplification technology according to claim 1, wherein in step (4), the high-performance liquid chromatography system uses a C18 reverse phase chromatographic column and a gradient elution mode.

9. The liquid chromatography method for simultaneously detecting multiple microRNAs based on a DSN cyclic amplification technology according to claim 8, wherein the gradient elution mode is that a proportion of methanol is changed from 10% to 60% in 20 min; and a mobile phase consists of an organic phase and an aqueous phase containing TEAA.

10. The liquid chromatography method for simultaneously detecting multiple microRNAs based on a DSN cyclic amplification technology according to claim 1, wherein processes of the method are all performed in a dark place.


SEQUENCE LISTING (3 sequences)

SEQ ID NO: 1 - DNA, 24 bp, Artificial Sequence
  It was synthesized.
tcaaacaccattgtcacactccac

SEQ ID NO: 2 - DNA, 25 bp, Artificial Sequence
  It was synthesized.
tacccctatcacgattagcattaat

SEQ ID NO: 3 - DNA, 24 bp, Artificial Sequence
  It was synthesized.
ttcaacatcagtctgataagctat